(19) United States
(12) Patent Application Publication
(10) Pub. No.: US 2005/0163822 A1
(43) Pub. Date: Jul. 28, 2005

(54) Biodegradable bio-absorbable material for clinical practice and method for producing the same

(76) Inventors: Hiroyuki Shirahama, Hiroshima (JP); Masamitsu Miyazaki, Aichi (JP); Mikio Fukuchi, Aichi (JP)

Correspondence Address: Barry E. Negrin;Levisohn, Berger & Langsam, LLP, 19th Floor, 805 Third Avenue, New York, NY, 10022, US

(21) Appl. No.: 11/020,535

(22) Filed: Dec. 22, 2004

Related U.S. Application Data
(63) Continuation of application No. PCT/JP02/06330, filed on Jun. 25, 2002.

(51) Int. Cl. A61K 38/12; A61K 31/785

(52) U.S. Cl. 424/426; 514/9; 424/78.27; 525/54.1

(57) ABSTRACT

Bio-absorbable polymers such as vascular stent and suture thread for use as materials for clinical practice have almost definite dynamic properties such as tensile strength and degradation rate for absorption. When the dynamic properties thereof are elevated, therefore, the bio-absorbable polymers turn fragile, involving slower degradation rate. When the degradation rate is elevated, further, the dynamic properties are deteriorated. Disadvantageously, such bio-absorbable polymers have limited purposes for use and limited sites for use. Thus, copolymerization of bio-absorbable polymers with a cyclic depsipeptide to form a copolymer of the ring-opened and copolymerized depsipeptide can allow the adjustment of the dynamic properties and degradation rate of the resulting copolymer depending on the content of the depsipeptide.

### TECHNICAL FIELD

The present invention relates to a biodegradable bio-absorbable material of a bio-absorbable polymer for clinical practice, which can be used for a medical device made of biodegradable bio-absorbable material, such as suture thread, vascular stent, biological cell carrier, and carriers of drug and the like, and a method for producing the same.

### BACKGROUND OF THE INVENTION

Bio-absorbable polymers for use as medical materials such as vascular stent and suture thread include for example polylactic acid, polyglycolic acid, a copolymer of the two, namely polyglactin, polydioxanone, and polyglyconate (the copolymer of trimethylene carbonate and glycolide).

Such bio-absorbable polymers are degraded and absorbed in biological organisms. Therefore, such bio-absorbable polymers are widely used. Because the dynamic properties thereof such as tensile strength and the degradation rate thereof for absorption are individually nearly definite, the bio-absorbable polymers turn fragile when the dynamic properties are enhanced, involving the reduction of the degradation rate. When the degradation rate is increased, alternatively, the dynamic properties are deteriorated. Thus, disadvantageously, the bio-absorbable polymers have only limited purposes for use and are applied to limited sites.

### DISCLOSURE OF THE INVENTION

The present invention relates to a biodegradable bio-absorbable material of a bio-absorbable polymer for clinical practice, which is a copolymer of a ring-opened and copolymerized depsipeptide as produced by copolymerizing together a bio-absorbable polymer and a cyclic depsipeptide so that the content of the depsipeptide can adjust the dynamic properties and degradation rate thereof, without any occurrence of inflammation and other problems.

The amount of the depsipeptide to be added is at about 2% to 60% in molar ratio. Below 2%, the effect thereof cannot be exerted. At 60% or more, the resulting dynamic properties are too much deteriorated. Many types of bio-absorbable polymers can be utilized. Depending on the type of a bio-absorbable polymer or the amount of a bio-absorbable copolymer to be blended, the amount of the depsipeptide to be added outside the limit range of the amount of the depsipeptide to be added as described above may sometimes involve the exertion of the effect. Therefore, the ratio of the amount thereof to be added is not a definite value.

### BRIEF DESCRIPTION OF THE DRAWINGS

FIG. 1 depicts the structure view of depsipeptide;

FIG.2 depicts the structure view of a tercopolymer with a depsipeptide unit;

FIG. 3 depicts the explanatory scheme of the synthesis of the depsipeptide;

FIG. 4 depicts the structure view of the tercopolymer of a ring-opened and copolymerized depsipeptide;

FIG. 5 depicts the chart of the ¹H-NMR spectrum of the tercopolymer;

FIG. 6 depicts the graphs of the hydrolysis tests with a degradation solution containing a buffer alone;

FIG. 7 depicts the graphs of the results of the enzymatic degradation profiles of the tercopolymers and each of the homopolymers with proteinase K;

FIG. 8 depicts the graphs of the degradation profiles of the copolymers with the depsipeptides;

FIG. 9 shows graphs depicting the relations among the amounts of the depsipeptides and the degradation rates;

FIG. 10 is a figure or table showing the synthetic conditions of each of the copolymers and homopolymers, the yields and molecular weights of the polymers;

FIG. 11 is a figure or table depicting the thermal properties of each of the copolymers and the homopolymers;

FIG. 12 is a figure or table depicting the mechanical properties (tensile strength) of the tercopolymers and the thermal properties thereof;

FIG. 13 is a figure or table depicting the changes of various physico-chemical properties of the tercopolymers before and after degradation with proteinase K; and

FIG. 14 is a figure or table depicting the relations among the amounts of depsipeptides and the thermal properties.

### BEST MODE FOR CARRYING OUT THE INVENTION

So as to describe the invention in more detail, the invention is now described with reference to the attached drawings.

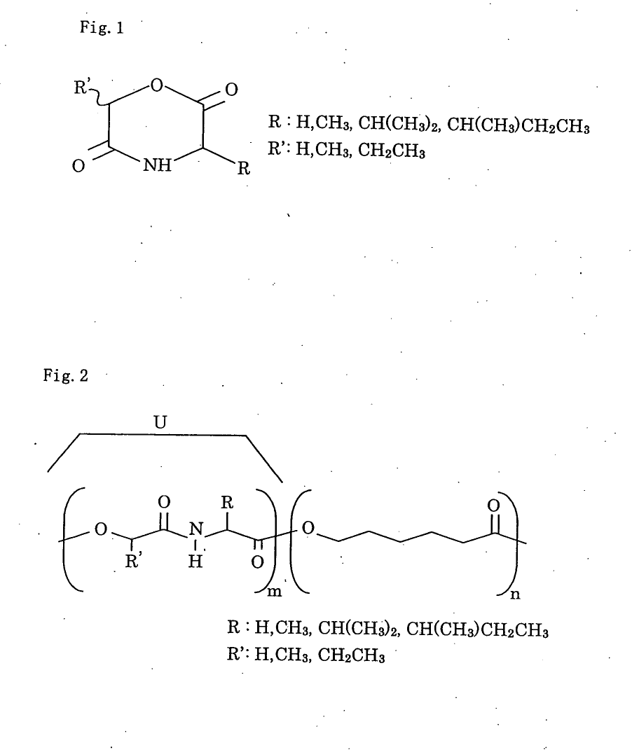

The structure of the depsipeptide is shown in FIG. 1.

As shown in the figure, the R group in a side chain is an alkyl group such as methyl group, isopropyl group and isobutyl group, while the R′ group in a side chain is an alkyl group such as methyl group and ethyl group.

Concerning examples of the depsipeptide, depsipeptides are synthesized from amino acid and a hydroxylate derivative, using chloroacetyl chloride, 2-bromopropionyl bromide and 2-bromo-n-butyryl bromide are as the hydroxylate derivative to prepare depsipeptides, namely L-MMO, L-DMO, and L-MEMO, in the order of the hydroxylate derivatives. All of them are applicable to the invention. The enzymatic degradation level of a copolymer from such depsipeptide monomer and a bio-absorbable polymer ε-caprolactone (CL) with proteinase K is in the order of L-MMO/CL >L-DMO/CL>L-MEMO/CL.

As to the depsipeptide synthesized from amino acid and an oxyacid derivative, amino acids such as L-alanine, L-(DL- or D-)valine, and L-leucine are used to prepare depsipeptides, namely DMO, PMO and BMO in the order of the amino acids. All of them are applicable to the invention. The enzymatic degradation level of a copolymer from such depsipeptide monomer and a bio-absorbable polymer ε-caprolactone (CL) with proteinase K is in the order of DMO/CL>PMO/CL≧BMO/CL. The enzymatic degradation level thereof with cholesterol esterase is in the order of PMO/CL>BMO/CL≧DMO/CL.

First Embodiment

A tercopolymer was prepared by adding a cyclic depsipeptide (DMO) to a copolymer of L-lactide (L-LA) as a raw material of polylactic acid and ε-caprolactone as a raw material of poly ε-caprolactone.

FIG. 2 is the structure view of the copolymer with the peptide unit as recovered by the polymerization of the depsipeptide. U expresses depsipeptide unit.

Therefore, 3,6-dimethyl-2,5-morpholine-dione (DMO) was synthetically prepared as a cyclic depsipeptide. The cyclic depsipeptide is a cyclic ester amide prepared from α-amino acid and a α-hydroxylate derivative. Herein, DL-alanine and DL-2-bromopropionyl bromide were used as α-amino acid and α-hydroxylate derivative, respectively.

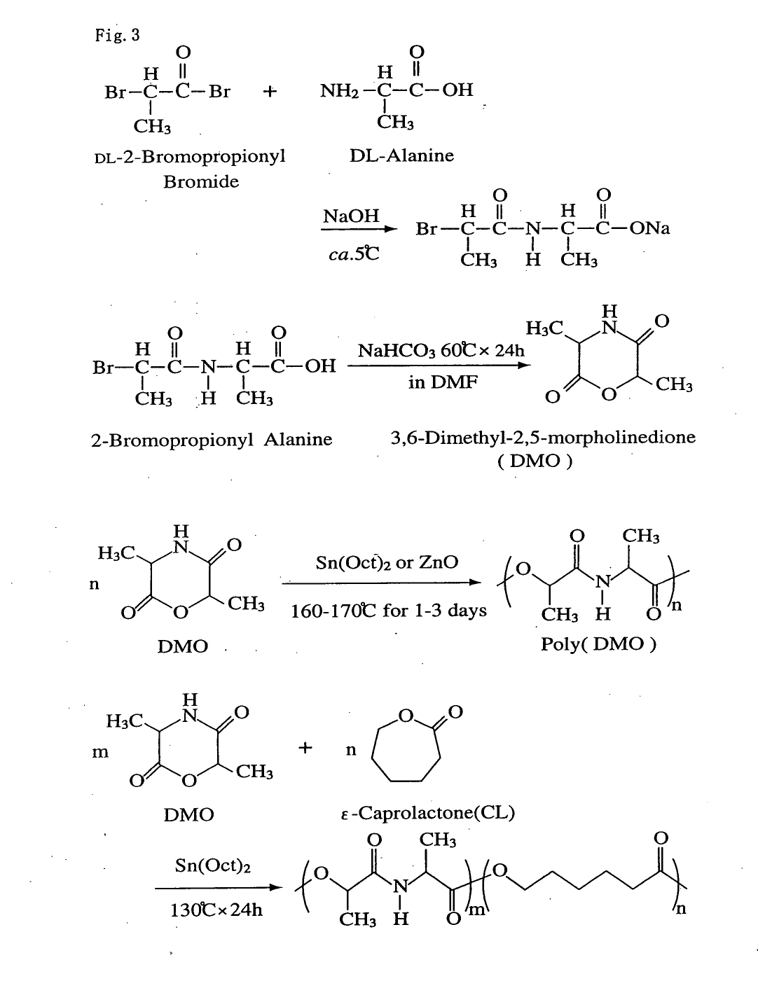

At the first step of the synthesis, the Schotten-Baumann reaction between alanine and 2-bromopropionyl bromide was carried out in an aqueous alkaline solution, for peptide linking to prepare 2-bromopropionyl alanine (FIG. 3).

In other words, 150 ml of an aqueous solution of DL-alanine (53.4 g; 0.6 mol) in 4N NaOH (0.6 mol) was cooled to about 5° C., to which were then added alternately 180 ml of 4N NaOH (0.72 mol) and 69.9 ml of DL-2-bromopropionyl bromide (0.66 mol) under cooling and agitation in an ice bath over about 30 minutes. The reaction mixture was continuously kept at mild alkalinity. After the termination of the reaction, the product in white was filtered and isolated.

The product was dissolved in water, followed by dropwise addition of 5N HCl to about pH 3. Thereafter, water was removed by evaporation. While the remaining aqueous solution was gradually acidified with 5N HCl under cooling, an additional product in white was recovered. These white products recovered were extracted in diethyl ether with a Soxhlet extractor, for purification.

Yield 30 to 40%; ¹H NMR(δ, CDCl₃) 1.54(d, 3H, NHCHCH₃), 1.91(d, 3H, BrCHCH₃), 4.45(q, 1H, NHCHCH₃), 4.59(q, 1H, BrCHCH₃), 6.88(brs, 1H, NH).

Continuously, the purified 2-bromopropionyl alanine (19.7 g; 0.0881 mol) and the equimolar NaHCO₃ (7.40 g; 0.0881 mol) were added to 150 ml of dimethylformamide (DMF). Then, the resulting mixture was refluxed at 60° C. for 24 hours, for intramolecular cyclization desalting, to recover a cyclic depsipeptide DMO in white powder (FIG. 3).

DMO was purified via recrystallization twice in chloroform.

Yield 40 to 60%; mp 158 to 159° C.; 1H NMR(δ CDCl₃) 1.54(d, 3H, NHCHCH₃), 1.62(d, 3H, OCHCH₃), 4.24(q, 1H, NHCH), 4.91(q, 1H, OCH), 7.07 ppm (brs, 1H, NH).

The synthesis of the tercopolymer is now described.

Among the copolymerizable monomers, the cyclic depsipeptide (L-DMO) was synthetically prepared from α-amino acid (L-alanine) and a α-hydroxylate derivative (DL-2-bromopropionyl bromide), and was then purified for use.

Further, lactone (CL) was purified by dissolving CL in toluene and subsequently drying CL with CaH₂ for 48 hours, and then subjecting the resulting CL to distillation under reduced pressure (twice). L-Lactide (L-LA) was purified by recrystallization in THF and sublimation (twice).

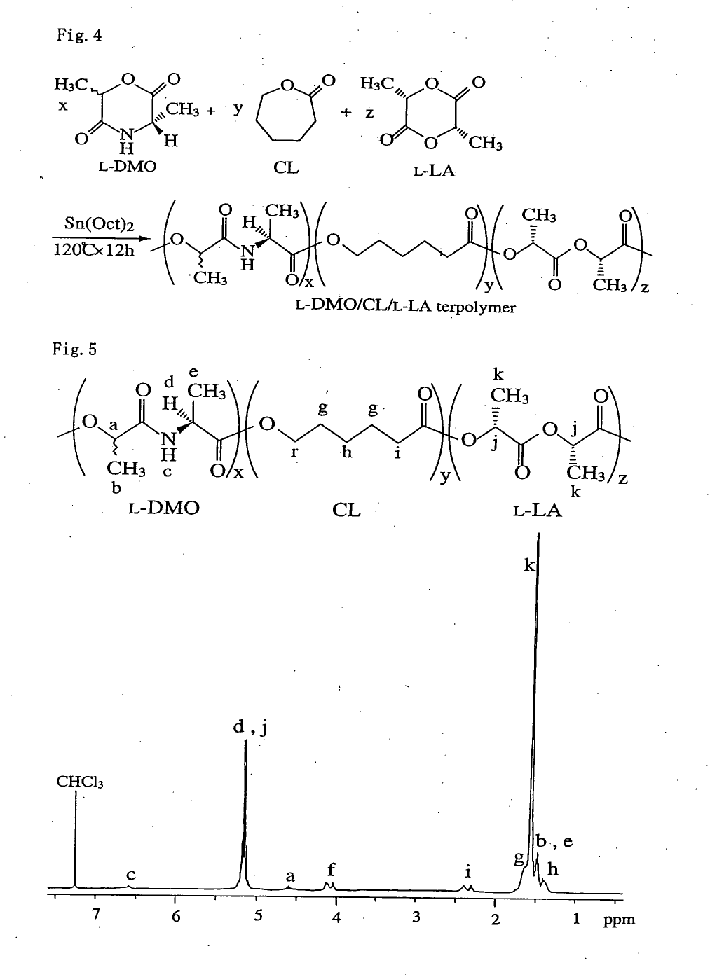

All the polymerization procedures were done in argon atmosphere. The synthetic scheme of the L-DMO/CL/L-LA tercopolymer is shown in FIG. 4.

The copolymer was prepared as follows.

Given amounts of both the monomers L-DMO and L-LA dissolved in THF and a toluene solution of a catalytic amount of tin (II) octylate [Sn (Oct)₂; 0.2 mol %/monomer] are charged in a Schrenk tube (polymerization container), from which the solvents THF and toluene are subsequently trapped and removed under reduced pressure.

Then, a given amount of the CL monomer is placed in the same polymerization container, which is then sealed. The sealed container was immersed in an oil bath at 120° C., to initiate the polymerization.

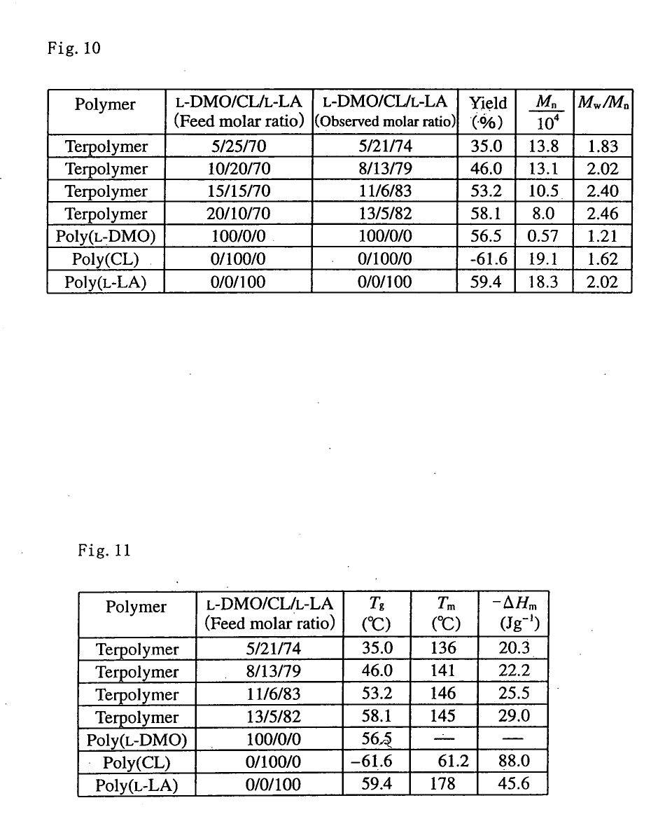

After a given period of time (12 hours), the polymerization container was taken out of the oil bath and then cooled. The resulting crude polymer was dissolved in chloroform and purified via reprecipitation (twice) in methanol. FIG. 10 shows the synthetic conditions of each of the copolymers and the homopolymers and the yields and molecular weights of the resulting polymers.

Further, the ¹H-NMR data (δ, CDCl₃) of the tercopolymer L-DMO/CL/L-LA (=8:13:79) is as follows.

1.38(m, 2H, CH₂CH₂CH₂CH₂CH₂), 1.50(m, 6H, CH₃×2(L-DMO)), 1.57(d, 6H, CH₃×2(L-LA) ), 1.68(m, 4H, CH₂CH₂CH₂CH₂CH₂), 2.25 to 2.45(splitting in two peaks, 2H, CCH₂), 4.60(m, 1H, OCH(L-DMO)), 5.17(q, 3H, OCH×2(L-LA), NHCH(L-DMO)), 6.60 ppm(br.m, 1H, NH). Various physico-chemical properties of the polymers are now described.

The composition of the copolymer was determined on the basis of the peak integration ratio of ¹H NMR spectrum measured with a 400 MHz magnetic resonance system (JEOL JMN-LA400). Based on the spectrum, the chain sequence (randomness) of the copolymer was deduced as well.

The number average molecular weight (Mn) of the polymer and the molecular distribution (Mw/Mn) thereof were determined on the basis of a standard curve prepared from standard polystyrene, using GPC 8010 system manufactured by TOSO, Co., Ltd. [column: TSK Gel (G2000H_{HR}+G3000H_{HR}+G4000H_{HR}+G5000H_{HR}), column temperature of 40° C. and differential refractive index (RI) meter]. Chloroform was used as the eluent at the flow of 1 mLmin¹.

The thermal properties of the polymer, namely the glass transition temperature (Tag), melting point (Tm) and melting heat (ΔHm) thereof were measured, using a differential scanning calorimeter SSC5100 DSC22C manufactured by Seiko Electric Co., Ltd. The measurement was done in nitrogen atmosphere at a temperature elevation rate of 10° C./min. Then, the randomness of the copolymer was also deduced on the basis of the values of these thermal properties.

The mechanical properties of the polymer (tensile strength and elongation at break) were measured, using a tensile tester RTC-1210 A manufactured by Orientec Co., Ltd. at a crosshead speed of 50 mm/min. The measurement was done at least at three times, to use the average. Additionally, the dumbbell test piece (parallel length×width×thickness=12×2.65×1.46 mm) of a polymer sample was prepared by pressing the polymer material under heating at 180 to 200° C. for about 5 minutes.

The enzymatic degradation test of the polymer is now described. The enzymatic degradation test was done in the same manner as in the related art. The test is now summarized below.

Polymer film (film thickness of about 100 μm; several tens milligrams) sealed in polyethylene sheet mesh (mesh size of about 1×1 mm) was incubated (37° C.) in a sample tube bottle placing an enzyme and a buffer (50 ml) therein, for degradation. The enzyme concentration was 1 International Unit (IU) per 1 mg of the polymer sample.

Herein, the buffer containing the enzyme (degradation solution) was exchanged to a fresh one every about 40 hours, taking account of the reduction of the oxygen activity and the contamination and growth of microorganisms in air.

The degradation level was evaluated on the basis of the changes of the weight and physico-chemical properties (molecular weight, composition and thermal properties) of the polymer before and after degradation. Proteinase K (derived from Tritirachium album; manufactured by Wako Pure Chemical Industries, Ltd.; activity of 20 IU/mg) was used as one of proteinases, while Tricine (pH 8.0) was used as Good's buffer.

The polymerization results of the tercopolymer L-DMO/CL/L-LA and each of the homopolymers from the aforementioned tests are shown in FIG. 10.

In case of the CL homopolymer and the L-LAhomopolymer [poly(CL) and poly (L-LA), respectively], the polymers of high molecular weights were recovered at higher yields. Only an L-DMO homopolymer [poly(L-DMO)] of a low molecular weight was recovered at a low yield. This may be due to the results of the ready occurrence of the ester exchange reaction of the resulting depsipeptide polymer with the monomer (herein, L-DMO) and the oligomers and of the back biting reaction thereof causing molecular chain break.

In case of the tercopolymer, alternatively, the reactivity of L-LA is high under the copolymerization conditions on the basis of the copolymer composition ratio.

Because L-DMO is incorporated more in the copolymer than CL, further, the reactivity of L-DMO is not so high as CL. Therefore, the reason why the molecular weight and yield of the L-DMO homopolymer are low may be ascribed to the ready occurrence of the ester exchange. The molecular weight and yield of the copolymer are relatively great, but the values thereof are gradually decreased as the content of L-DMO is increased. This also supports the consideration.

The thermal properties of the resulting tercopolymer and each of the homopolymers are shown in FIG. 11.

As traditionally reported, poly(CL) has softness such that Tg and Tm thereof are about −60° C. and 60° C., respectively. However, a crystallizable polymer with a low melting point, namely poly(L-LA) has Tg and Tm at about 60° C. and 180° C., respectively. The poly(L-LA) is so rigid and fragile crystallizable polymer with a high melting point, while poly(L-DMO) is an amorphous glass-like polymer.

In case of the tercopolymer, single one Tg and single one Tm are only observed and their values change as the composition changes, suggesting intense randomness. From the respect of balance between the tensile strength and elongation (softness) of the mechanical properties, appropriately, the Tg value may be around 35° C.

FIG. 5 depicts the ¹H NMR spectrum of the tercopolymer L-DMO/CL/L-LA (8:13:79). The chart establishes the verification that the tercopolymer is random. In other words, the proton peaks (f, I) of the α- and ε-methylene in the CL unit are sensitive to the adjacent comonomer units. It is indicated that because these peaks are individually split into two (the peak on the side of high magnetic field corresponds to the homosequence of CL-CL; the peak on the side of low high magnetic field correspond to a peak based on the hetero-sequence of L-LA-CL and L-DMO-CL), the tercopolymer is a random copolymer.

Additionally, the reason why the unit L-DMO is introduced appropriately in the resulting copolymer via copolymerization at 120° C. lower than the Tm thereof (about 170° C.) is that the polymerization of the highly reactive L-LA (with Tm of about 95° C.) first occurs and the active elongating terminus induces the ring-opening of the L-DMO (and/or CL), which is then incorporated randomly in the copolymer.

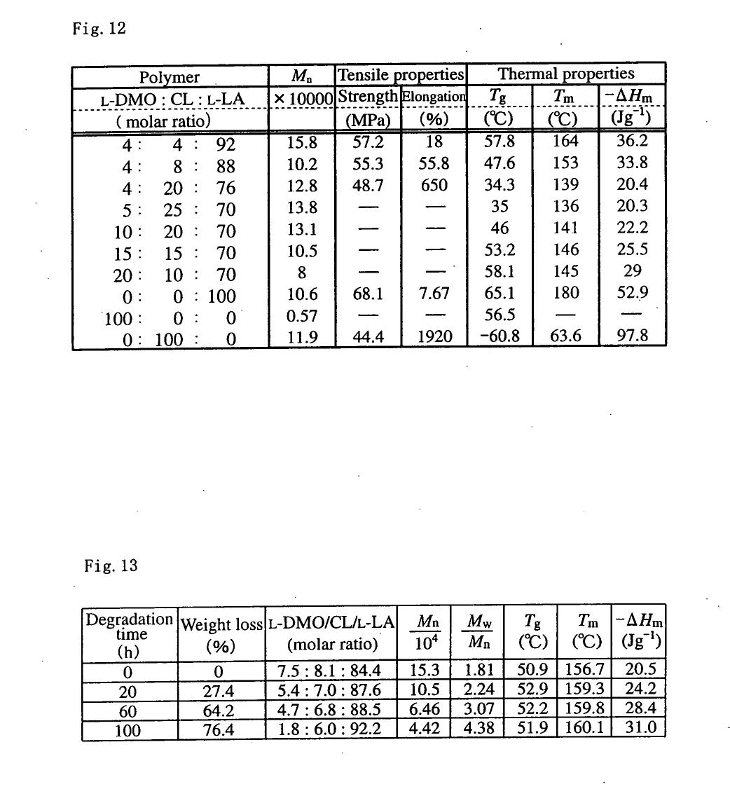

FIG. 12 depicts the mechanical properties (tensile profile) and thermal properties of the tercopolymer.

The tercopolymer in the figure was synthetically prepared freshly at a large scale, so as to measure these physico-chemical properties. (So as to modify the softness of the polymer, mainly, the tercopolymer has been synthetically prepared, while changing the CL amount.) From the respect of molecular weight (Mn), all the resulting copolymers had molecular weights of 100,000 or more (102,000 to 158,000). It is therefore not so much required to consider the influence of the molecular weight on the physico-chemical properties.

As to the tensile profile, first, the tensile strength is reduced as the CL content increases or the L-LA amount decreases. Alternatively, the elongation rapidly increases at the CL amount of 20 mol % or more, which indicates that the softness of the copolymer is improved.

The tensile strengths of these tercopolymers are larger than that of a common plastic polyethylene (PE) and are of values equal to or larger than the value of polypropylene (PP). The tensile strengths thereof are larger than those of biodegradable plastics Bionolle [polybutylene succinate (PBSU); manufactured by Showa Polymer Co., Ltd.] and Biopol [P(3HB-co-3HV); manufactured by Nippon Monsanto Co., Ltd.].

The break elongation of a sample at a CL content of 20 mol % or more is far larger than that of Biopol and is at the same level as or a higher level than those of PE, PP and Bionolle. Like the case in FIG. 11., alternatively, both the thermal properties Tm and Δ Hm of the tercopolymers are decreased when the CL amount is increased. It is shown that all the tercopolymers are crystallizable polymers with Tm of 100° C. or more.

Judging from these mechanical properties and thermal properties, the tercopolymer L-DMO/CL/L-LA (4:20:76) (Tg=34.3° C.) at a CL content of 20 mol% or more has a good balance in the physico-chemical properties.

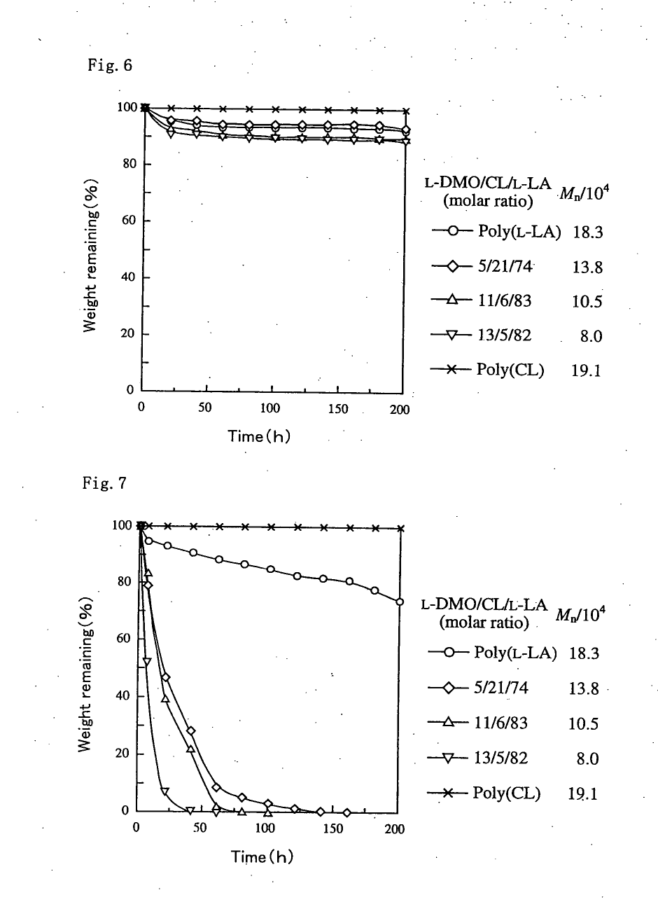

Before examination of the enzymatic degradation level, then, a hydrolysis test with a degradation solution containing a buffer alone was done (FIG. 6). Herein, the L-DMO homopolymer was not used at the hydrolysis test because the homopolymer was water-soluble. As shown in the figure, the hydrolysis level is decreased as the CL unit amount is increased but is alternatively increased as the L-DMO unit is increased. Thus, the increasing order of hydrophobicity is L-DMO<L-LA<CL unit. However, the weight loss of a tercopolymer at the maximum is about 10% 200 hours later.

FIG. 7 shows the oxygen degradation levels of the tercopolymer and each of the homopolymers with proteinase K.

In case of the homopolymers, poly(L-LA) is decomposed at some extent. Poly(CL) more readily decomposable from the respect of thermal properties hardly lost the weight within 200 hours. This may be ascribed to the high substrate specificity of the enzyme to polymers with shorter alkyl chain lengths between ester bonds and with side chains [herein, poly(L-LA) with lactoyl group {—O—CH(CH₃)—CO—}] but no specificity thereof to poly(CL) with the linear ethylene chain between ester bonds of a relatively long length. In case of the tercopolymer, alternatively, the degradation level is more increased compared with the poly(L-LA), when the content of the L-DMO unit is increased. The substrate specificity of the enzyme to the L-DMO unit with lactoyl group alike may be a big factor for this increase.

It is suggested as an additional cause that the molecular weight (Mn) and thermal properties (Tm, Δ Hm) of the tercopolymer are reduced, compared with poly(L-LA) (FIG. 11). In any way, the tercopolymer L-DMO/CL/L-LA could get an improved enzyme degradation level with proteinase K while the tercopolymer L-DMO/CL/L-LA relatively retained the thermal and mechanical properties of poly(L-LA).

So as to speculate the degradation mechanism with the enzyme, continuously, the changes of various physico-chemical properties of the tercopolymer L-DMO/CL/L-LA (8:8:84) before and after degradation with proteinase K were examined (FIG. 13). As shown in FIG. 13, the result is that the content of the L-DMO unit in the residual polymer was highly reduced as the degradation proceeded. As described above, the enzyme has substrate specificity to both the units L-LA and L-DMO. Because the polymer domain containing the L-LA unit is crystallizable, the degradation level of the amorphous hydrophilic region highly containing the L-DMO unit is elevated, possibly leading to the decrease of the composition ratio.

It is also suggested that because the amount of the CL unit hardly decomposable was also decreased, the CL unit existed adjacent to the L-DMO unit and the ester bond between the two units was broken with the enzyme. The molecular weight (Mn) of the polymer decreased as the degradation proceeded, while the distribution (Mw/Mn) was likely to be enlarged. Thus, it is indicated that the degradation with the enzyme randomly progressed in the inside of the polymer film.

Because the thermal properties (Tm, Δ Hm) of the polymer were elevated as the progress of the degradation, further, it is indicated that the degradation of the amorphous hydrophilic region highly containing the L-DMO unit preferentially occurred. Based on the results with NMR nuclear (magnetic resonance system) and the measurement of the thermal properties, it was shown that the resulting copolymer was a random copolymer.

This indicates that the degradation rate remarkably increased via the addition of depsipeptide, without any loss of the mechanical strength and softness.

Further, FIG. 7 shows the enzyme degradation properties of the copolymer with the depsipeptide unit.

Additionally, the above description has been done, provided that the lactide is L-lactide. L-Lactide and the enantiomer D-lactide are polymerized together in combination, to form a stereo complex with improved thermal properties such as melting point.

Still further, the change of the glass transition temperature can impart free formation potency.

Therefore, a bicopolymer produced by copolymerizing together a depsipeptide and L-lactide to ring-open and polymerize the depsipeptide may be satisfactory, other than the tercopolymer. Additionally, a lactide with a formed stereo complex as produced by copolymerizing a combination of L-lactide and the enantiomer D-lactide together is copolymerized with a depsipeptide, to form a stereo complex of a copolymer of the ring-opened and polymerized depsipeptide.

Second Embodiment

In case of the recovery of the copolymer of a depsipeptide ring-opened and polymerized, resulting from the copolymerization of a raw material of polyε-caprolactone, namely ε-caprolactone, the structure of the copolymer with the peptide unit is shown in FIG. 2. U expresses the depsipeptide unit. The procedure also imparted mechanical strength and increased degradation rate as in the first embodiment.

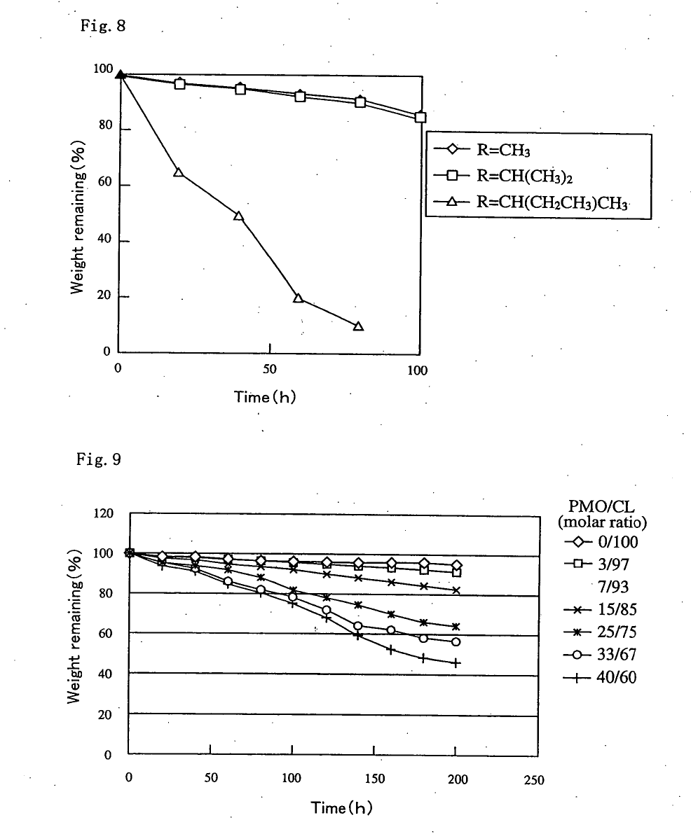

So as to elucidate the influence of the depsipeptide unit in the copolymer with the peptide units, further, the R group in the side chain in the depsipeptide was modified into methyl group, isopropyl group or isobutyl group, to examine the influence. FIG. 8 shows the degradation levels of the copolymers with the depsipeptide units.

The figure shows that the degradation level is in the order of methyl group >>isopropyl group >isobutyl group. It is shown that the increase of the bulkiness of the side chain decreases the degradation level.

Third Embodiment

3-Isopropyl-6-methyl-2,5-morpholine-dione (PMO) used as a depsipeptide was copolymerized with poly ε-caprolactone, to prepare a copolymer, where the depsipeptide was ring-opened and polymerized.

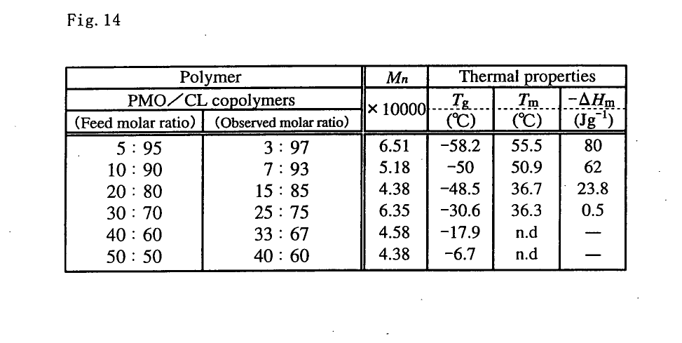

Then, the changes of the thermal properties and degradation rate in case of the change of the depsipeptide amount were examined. FIG. 14 shows the relation of the thermal properties, while FIG. 9 shows the relation of the degradation rate.

According to the results, the glass transition temperature (Tg) was elevated as the depsipeptide amount increased. At the amount of ε-caprolactone at 20 mol% or less, the melting point (Tm) and the melting heat (Δ Hm) were observed, indicating that the resulting copolymer was crystallizable.

The degradation rate was elevated as the amount of the depsipeptide increased.

Herein, the description in the individual embodiments has been done, exemplifying poly ε -caprolactone and polylactic acid as the bio-absorbable polymers. However, the bio-absorbable polymers are not limited to them. Any bio-absorbable polymer may be satisfactory, including for example polydioxanone, trimethylene carbonate and copolymers of two or more thereof.

Industrial Applicability

In accordance with the invention described in detail above, a copolymer with a depsipeptide unit, as produced by copolymerizing a cyclic depsipeptide with a bio-absorbable polymer, can advantageously be modified into a biodegradable bio-absorbable material with adjusted dynamic properties and degradation properties for clinical practice.

Further, advantageously, the modification of the peptide unit with alkyl groups can adjust the dynamic properties and the degradation properties.

1. A biodegradable bio-absorbable material for clinical practice, which is a copolymer produced by copolymerizing a bio-absorbable polymer and a cyclic depsipeptide together to ring-open and copolymerize the depsipeptide.

2. A biodegradable bio-absorbable material for clinical practice according to claim 1, where the amount of the depsipeptide is at 2 to 60%.

3. A biodegradable bio-absorbable material for clinical practice according to claim 1, where the bio-absorbable polymer is caprolactone.

4. A biodegradable bio-absorbable material for clinical practice according to claims 1, where the depsipeptide is a cyclic depsipeptide and the caprolactone is ε-caprolactone.

5. A biodegradable bio-absorbable material for clinical practice according to claim 1, where the bio-absorbable polymer is lactide.

6. A biodegradable bio-absorbable material for clinical practice according to claims 1, where the depsipeptide is a cyclic depsipeptide and the lactide is L-lactide.

7. A biodegradable bio-absorbable material for clinical practice according to claims 1, where the depsipeptide is a cyclic depsipeptide and the lactide is a stereo complex of L-lactide and D-lactide.

8. A biodegradable bio-absorbable material for clinical practice according to claim 1, where the bio-absorbable polymer is produced by copolymerizing together caprolactone and lactide.

9. A biodegradable bio-absorbable material for clinical practice according to claim 1, where the ratio of the depsipeptide and the bio-absorbable polymer is modified to adjust the biodegradation rate.

10. A biodegradable bio-absorbable material for clinical practice according to claim 1, where the R group in the side chain of the cyclic depsipeptide is an alkyl group.

11. A biodegradable bio-absorbable material for clinical practice according to claim 1, where the R′ group in the side chain of the cyclic depsipeptide is an alkyl group.

12. A biodegradable bio-absorbable material for clinical practice according to claim 1, where the R group in the side chain of the cyclic depsipeptide is methyl group.

13. A biodegradable bio-absorbable material for clinical practice according to claim 1, where the R group in the side chain of the cyclic depsipeptide is isopropyl group.

14. A biodegradable bio-absorbable material for clinical practice according to claim 1, where the R group in the side chain of the cyclic depsipeptide is isobutyl group.

15. A biodegradable bio-absorbable material for clinical practice according to claim 1, where the R′ group in the side chain of the cyclic depsipeptide is methyl group.

16. A biodegradable bio-absorbable material for clinical practice according to claim 1, where the R′ group in the side chain of the cyclic depsipeptide is ethyl group.

17. A method for producing a biodegradable bio-absorbable material for clinical practice, including ring-opening reaction of a cyclic depsipeptide during the copolymerization of a bio-absorbable polymer and the depsipeptide.
